(19) United States
(12) Patent Application Publication
(10) Pub. No.: US 2025/0075005 A1
(43) Pub. Date: Mar. 6, 2025

(54) USE OF ALPHA-ENOLASE ANTAGONIST IN TREATING OF FIBROTIC DISEASES

(71) Applicant: HUNILIFE BIOTECHNOLOGY, INC., Taipei (TW)

(72) Inventors: TA-TUNG YUAN, Taipei (TW); WEI-CHING HUANG, Taipei (TW); I-CHE CHUNG, Taipei (TW); CHI-FEN CHUANG, Taipei (TW); MAO-LIN CHEN, Taipei (TW); YUNG-TSANG HUANG, Taipei (TW)

(21) Appl. No.: 18/292,352

(22) PCT Filed: Aug. 19, 2022
(86) PCT No.: PCT/CN2022/113767

§ 371 Date: Jan. 26, 2024

Related U.S. Application Data
(60) Provisional application No. 63/235,486, filed on Aug. 20, 2021.

(51) Int. Cl. C07K 16/40; A61K 39/00; A61P 11/00

(52) U.S. Cl. CPC C07K 16/40; A61P 11/00; A61K 2039/505; C07K 2317/24

(57) ABSTRACT

Provided is the use of an effective amount of alpha-enolase (enolase-1, ENO-1) antagonist in manufacturing a medicament for treating a fibrotic disease. ENO-1 antagonist significantly attenuated body weight loss and lung weight gain as well as the fibrosis lesion and collagen deposition in lungs. Also, ENO-1 antagonist significantly reduced cell migration and secretion of collagen and TGF-β in primary mouse lung myofibroblasts.

### CROSS-REFERENCE TO RELATED APPLICATION

This application claims the benefit of U.S. Provisional Application No. 63/235,486, filed Aug. 20, 2021, which is hereby incorporated by reference herein for all purposes.

### BACKGROUND OF INVENTION

### Field of the Invention

The present disclosure is in the field of fibrotic diseases and connective tissue disorders. More specifically, the disclosure relates to the use of alpha-enolase (enolase-1, ENO-1) antagonist for the treatment and/or prevention of fibrotic diseases, in particular idiopathic pulmonary fibrosis.

### Background Art

Fibrotic diseases involve the formation of excess fibrous connective tissue in an organ or tissue in a reparative or reactive process. Such fibrotic diseases include idiopathic pulmonary fibrosis (IPF), pulmonary hypertension, pulmonary fibrosis, emphysema, nonalcoholic steatohepatitis, pancreatic fibrosis, intestinal fibrosis, cardiac fibrosis, myelofibrosis, arthrofibrosis, interstitial lung diseases, non-specific interstitial pneumonia (NSIP), usual interstitial pneumonia (UIP), endomyocardial fibrosis, mediastinal fibrosis, retroperitoneal fibrosis, progressive massive fibrosis (a complication of coal workers' pneumoconiosis), nephrogenic systemic fibrosis, Crohn's disease, old myocardial infarction, scleroderma/systemic sclerosis, neurofibromatosis, Hermansky-Pudlak syndrome, diabetic nephropathy, renal fibrosis, hypertrophic cardiomyopathy (HCM), hypertension-related nephropathy, focal segmental glomerulosclerosis (FSGS), radiation-induced fibrosis, uterine leiomyomas (fibroids), alcoholic liver disease, hepatic steatosis, hepatic fibrosis, hepatic cirrhosis, hepatitis C virus (HCV) infection, chronic organ transplant rejection, fibrotic conditions of the skin, keloid scarring, Dupuytren contracture, Ehlers-Danlos syndrome, epidermolysis bullosa dystrophica, oral submucous fibrosis, and fibro-proliferative disorders.

ENO-1 is a multiple functional protein, which was first found as a key enzyme of the glycolysis pathways. Under normal conditions, ENO-1 is expressed in the cytosol. However, ENO-1 is also found to express on the cell surfaces of many cancer cells as a plasminogen receptor and on activated hematopoietic cells, such as neutrophils, lymphocytes, and monocytes. It is known that the up-regulation of plasminogen receptor proteins can induce a cascade response of the urokinase plasminogen activation system and results in extracellular matrix degradation.

### SUMMARY OF INVENTION

The disclosure relates to an alpha-enolase (enolase-1, ENO-1) antagonist targeting ENO-1 and a use thereof, wherein the ENO-1 antagonist has a binding capacity to ENO-1, e.g. human ENO-1 antibody, as an antigen binding structural domain so as to neutralize the biological effect of the ENO-1. The ENO-1 antagonist can bind to free ENO-1 protein and ENO-1 protein on the surface of a cell and has an important application prospect in the treatment of fibrotic diseases.

In accordance with certain embodiments of the disclosure, the fibrotic diseases or disorder may be any condition arising from aberrant activation or expression of ENO-1 protein. Examples of such diseases include liver, gut, kidney, skin, epidermis, endodermis, muscle, tendon, cartilage, heart, pancreas, lung, uterus, nervous system, testis, penis, ovary, adrenal gland, artery, vein, colon, intestine (e.g. small intestine), biliary tract, soft tissue (e.g. mediastinum or retroperitoneum), bone marrow, joint, eye, stomach fibrosis or a combination thereof.

In accordance with certain embodiments of the disclosure, the fibrotic diseases or disorder may include idiopathic pulmonary fibrosis (IPF), pulmonary hypertension, pulmonary fibrosis, emphysema, nonalcoholic steatohepatitis, pancreatic fibrosis, intestinal fibrosis, cardiac fibrosis, myelofibrosis, arthrofibrosis, interstitial lung diseases, non-specific interstitial pneumonia (NSIP), usual interstitial pneumonia (UIP), endomyocardial fibrosis, mediastinal fibrosis, retroperitoneal fibrosis, progressive massive fibrosis (a complication of coal workers' pneumoconiosis), nephrogenic systemic fibrosis, Crohn's disease, old myocardial infarction, scleroderma/systemic sclerosis, neurofibromatosis, Hermansky-Pudlak syndrome, diabetic nephropathy, renal fibrosis, hypertrophic cardiomyopathy (HCM), hypertension-related nephropathy, focal segmental glomerulosclerosis (FSGS), radiation-induced fibrosis, uterine leiomyomas (fibroids), alcoholic liver disease, hepatic steatosis, hepatic fibrosis, hepatic cirrhosis, hepatitis C virus (HCV) infection, chronic organ transplant rejection, fibrotic conditions of the skin, keloid scarring, Dupuytren contracture, Ehlers-Danlos syndrome, epidermolysis bullosa dystrophica, oral submucous fibrosis, and fibro-proliferative disorders. In a preferred embodiment, the fibrotic diseases or disorder may include idiopathic pulmonary fibrosis, pulmonary hypertension, emphysema, nonalcoholic steatohepatitis, pancreatic fibrosis, renal fibrosis, intestinal fibrosis, cardiac fibrosis, myelofibrosis, arthrofibrosis, or systemic sclerosis.

In accordance with certain embodiments of the disclosure, the ENO-1 antagonist may be an anti-ENO-1 antibody or the binding fragment thereof. In accordance with certain embodiments of the disclosure, the ENO-1 antagonist may be an anti-ENO-1 chimeric antigen receptor (CAR), comprising an antigen binding domain, a hinge region, a transmembrane domain, and a signaling domain; wherein the antigen binding domain is at least a portion of anti-ENO-1 antibody.

In accordance with certain embodiments of the disclosure, the antigen binding domain may comprise the amino acid sequences shown in SEQ ID NO: 1 (GYTFTSCVMN), SEQ ID NO: 2 (YINPYNDGTKYNEKFKG), SEQ ID NO: 3 (EGFYYGNFDN), SEQ ID NO: 4 (RASENIYSYLT), SEQ ID NO: 5 (NAKTLPE) and SEQ ID NO: 6 (QHHYGTPYT). Preferably, the antibody may comprise a heavy-chain variable domain having three complementary regions including HCDR1 (GYTFTSCVMN; SEQ ID NO: 1), HCDR2 (YINPYNDGTKYNEKFKG; SEQ ID NO: 2), and HCDR3 (EGFYYGNFDN; SEQ ID NO: 3); and a light-chain variable domain having three complementary regions including LCDR1 (RASENIYSYLT; SEQ ID NO: 4), LCDR2 (NAKTLPE; SEQ ID NO: 5), and LCDR3 (QHHYGTPYT; SEQ ID NO: 6).

In accordance with other embodiments of the disclosure, the antigen binding domain may comprise the amino acid sequences shown in SEQ ID NO: 7 (GYTFTSXVMN, wherein X is any amino acid but cysteine), SEQ ID NO: 2 (YINPYNDGTKYNEKFKG), SEQ ID NO: 3 (EGFYYGNFDN), SEQ ID NO: 4 (RASENIYSYLT), SEQ ID NO: 5 (NAKTLPE) and SEQ ID NO: 6 (QHHYGTPYT). Preferably, the antibody may comprise a heavy-chain variable domain having three complementary regions including HCDR1 (GYTFTSXVMN, wherein X is any amino acid but cysteine; SEQ ID NO: 7), HCDR2 (YINPYNDGTKYNEKFKG; SEQ ID NO: 2), and HCDR3 (EGFYYGNFDN; SEQ ID NO: 3); and a light-chain variable domain having three complementary regions including LCDR1 (RASENIYSYLT; SEQ ID NO: 4), LCDR2 (NAKTLPE; SEQ ID NO: 5), and LCDR3 (QHHYGTPYT; SEQ ID NO: 6).

In accordance with certain embodiments of the disclosure, the hinge region may comprise a CD8 alpha hinge region (TTTPAPRPPTPAPTIASQPLSLRPEACRPAAGGAVHT RGLDFACD; SEQ ID NO: 8); optionally, the transmembrane domain may comprise a CD8 alpha transmembrane region (IYIWAPLAGTCGVLLLSLVIT; SEQ ID NO: 9) and/or a CD28 transmembrane region (FWVLVVVGGVLACYSLLVTVAFIIFWV; SEQ ID NO: 10); optionally, the signaling domain may comprise CD3 zeta (RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQALPP R; SEQ ID NO: 11); optionally, the signaling domain may further comprise 4-1BB, the intracellular region of CD28, DAP10, OX 40 or a combination thereof; optionally, the anti-ENO-1 CAR may further comprise a signal peptide; optionally, the signal peptide may comprise an IgG kappa light chain signal peptide, a CD8 alpha signal peptide, a GM-CSF signal peptide, an HSA signal peptide, an IgG heavy chain signal peptide, an IgG light chain signal peptide, a CD33 signal peptide, an IL-2 signal peptide, or an insulin signal peptide.

In accordance with certain embodiments of the disclosure, the anti-ENO-1 CAR may comprise a signal peptide, an anti-ENO-1 antibody, a CD8 alpha hinge region, a CD8 alpha transmembrane region, 4-1BB, and CD3 zeta.

In accordance with certain embodiments of the disclosure, the ENO-1 antagonist may be an immunoconjugate that binds specifically to ENO-1, comprising: the general formula of Ab-(L-D)m (I), wherein Ab is an anti-ENO-1 antibody or the binding fragment thereof, L is a linker or a direct bond, D is a therapeutic agent or a label, and m is an integer from 1 to 12. Preferably, the antibody may be a monoclonal antibody. Preferably, the antibody may be a mouse antibody, a human antibody, a chimeric antibody, a humanized antibody, or an antibody fragment thereof.

In accordance with certain embodiments of the disclosure, the therapeutic agent may comprise an anti-fibrotic agent, immunomodulator, radioactive isotopes, and toxins. Preferably, the ENO-1 antagonist can be used in combination with at least one therapeutically active agent with known anti-fibrotic activity selected from pirfenidone or receptor tyrosine kinase inhibitors (RTKIs) such as Nintedanib, Sorafenib and other RTKIs, or angiotensin II (AT1) receptor blockers, or CTGF inhibitor, or any antifibrotic compound susceptible to interfere with the TGF-β and BMP-activated pathways including activators of the latent TGF-β complex such as MMP2, MMP9, THBS1 or cell-surface integrins, TGF3 receptors type I (TGFBRI) or type II (TGFBRII) and their ligands such as TGF3, Activin, inhibin, Nodal, anti-Mijllerian hormone, GDFs or BMPs, auxiliary co-receptors (also known as type III receptors), or components of the SMAD-dependent canonical pathway including regulatory or inhibitory SMAD proteins, or members of the SMAD-independent or non-canonical pathways including various branches of MAPK signaling, TAK1, Rho-like GTPase signaling pathways, phosphatidylinositol-3 kinase/AKT pathways, TGF-β-induced EMT process, or canonical and non-canonical Hedgehog signaling pathways including Hh ligands or target genes, or any members of the WNT, or Notch pathways which are susceptible to influence TGF-β signaling.

In accordance with certain embodiments of the disclosure, the ENO-1 antagonist can be administered by oral, parenteral, buccal, vaginal, rectal, inhalation, insufflation, sublingual, intramuscular, subcutaneous, topical, intranasal, intraperitoneal, intrathoracic, intravenous, epidural, intrathecal, or intracerebroventricular route, or by injection into joint.

In accordance with certain embodiments of the disclosure, the subject is a mammal. In a preferred embodiment the subject is human.

In accordance with certain embodiments of the disclosure, the ENO-1 antagonist can be a nucleic acid, e.g. DNA or RNA, designed to be delivered into cells and expressed as intracellular protein of peptide. For example, the ENO-1 antagonist can be a nucleic acid that can be transcribed and translated as, for instance, an anti-ENO-1 antibody or the binding fragment thereof. In addition, the nucleic acid can be with or without a secretion signal peptide so that the proteins or the peptides transcribed and translated from the nucleic acid can be secreted, non-secreted or a combination thereof. In accordance with certain embodiments of the disclosure, the nucleic acid can be delivered to the subject via any known methods or medium, e.g. viral vector, polymer, or liposome. In accordance with certain embodiments of the disclosure, the nucleic acid may be substituted with known modified nucleotides, e.g. Pseudo UTP, 1-Me pseudo UTP, 5-Methoxy UTP, N1-Ethyl pseudo UTP, 5-Methyl CTP or N4-Acetyl-CTP, to enhance the expression efficiency.

Compared with the prior art, the disclosed inventions have the following beneficial effects. According to the present disclosure, in vivo and in vitro experiments demonstrate the anti-fibrotic efficacy and the anti-inflammatory efficacy of the ENO-1 antagonist, e.g. ENO-1 mAb HuL217. That is, the ENO-1 antagonist is a potential therapy for the fibrotic diseases, e.g. IPF.

One skilled in the art would appreciate that the pharmaceutically effective amount depends on many factors, such as patient conditions, age, disease states, routes of administration, etc., and that such effective amount may be determined based on these factors in routine practice without undue experimentation.

Other aspect of the disclosed invention will become apparent with the following description.

### BRIEF DESCRIPTION OF THE DRAWINGS

FIG. 1 shows overexpression of ENO-1 in human fibrotic lungs and lungs from murine model of bleomycin-induced lung fibrosis. The details are described in Example 2.

FIG. 2 shows in vivo anti-fibrotic efficacy of HuL217 in reducing body weight loss and lung weight gain in murine model of bleomycin-induced lung fibrosis. The details are described in Example 3.

FIG. 3 shows in vivo anti-fibrotic efficacy of ENO-1 mAb HuL217 in reducing Ashcroft score and inflammation score in the lung sections from murine model of bleomycin-induced lung fibrosis. The details are described in Example 3.

FIG. 4 shows in vivo anti-fibrotic efficacy of ENO-1 mAb HuL217 in reducing collagen in the lungs and TGF-β in bronchoalveolar lavage fluid from murine model of bleomycin-induced pulmonary fibrosis. The details are described in Example 3.

FIG. 5 shows in vivo anti-fibrotic efficacy of ENO-1 mAb HuL217 in reducing myofibroblasts (α-SMA positive) in the lungs from murine model of bleomycin-induced pulmonary fibrosis. The details are described in Example 3.

FIG. 6 shows in vivo anti-inflammatory efficacy of ENO-1 mAb HuL217 in reducing recruitment of monocytes and neutrophils in the lungs from murine model of bleomycin-induced pulmonary fibrosis. The details are described in Example 3.

FIG. 7 shows in vitro anti-fibrotic efficacy of ENO-1 mAb HuL217 in reducing migration of TGF-3-stimulated primary mouse and human lung fibroblasts. The details are described in Example 4.

FIG. 8 shows in vitro anti-fibrotic efficacy of ENO-1 mAb HuL217 in reducing collagen, TGF-01, and VEGF secretion in TGF-β 1-stimulated primary human normal fibroblasts. The details are described in Example 4.

FIG. 9 shows in vitro anti-fibrotic efficacy of ENO-1 mAb HuL217 in reducing collagen, TGF-01, and VEGF secretion in primary human IPF fibroblasts. The details are described in Example 4.

### DETAILED DESCRIPTION

### General Definitions

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press, 1989); DNA Cloning, Volumes I and II (D. N. Glover ed., 1985); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Antibodies: A Laboratory Manual, by Harlow and Lane s (Cold Spring Harbor Laboratory Press, 1988); and Handbook Of Experimental Immunology, Volumes I-IV (D. M. Weir and C. C. Blackwell, eds., 1986).

The terms “antibody” and “immunoglobulin” are used interchangeably in the broadest sense and include monoclonal antibodies (e.g., full length or intact monoclonal antibodies), polyclonal antibodies, monovalent, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

The antibodies can be full-length or can comprise a fragment (or fragments) of the antibody having an antigen-binding portion, including, but not limited to, Fab, F(ab′)₂, Fab′, F(ab)′, Fv, single chain Fv (scFv), bivalent scFv (bi-scFv), trivalent scFv (tri-scFv), Fd, dAb fragment (e.g., Ward et al, Nature, 341:544-546 (1989)), an CDR, diabodies, triabodies, tetrabodies, linear antibodies, single-chain antibody molecules, and multispecific antibodies formed from antibody fragments. Single chain antibodies produced by joining antibody fragments using recombinant methods, or a synthetic linker, are also encompassed by the present invention. Bird et al. Science, 1988, 242:423-426. Huston et al, Proc. Natl. Acad. Sci. USA, 1988, 85:5879-5883.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing or decreasing inflammation and/or tissue/organ damage, decreasing the rate of disease progression, amelioration or palliation of the disease state, and remission or improved prognosis. In some embodiments, antibodies of the present disclosure are used to delay development of a disease or disorder.

An “individual” or a “subject” is a vertebrate. In certain embodiments, the vertebrate is a mammal. Mammals include, but are not limited to, farm animals (such as cows), sport animals, pets (such as cats, dogs, and horses), primates, mice, and rats. In certain embodiments, the vertebrate is a human.

An “effective amount” refers to an amount effective, at dosages and for periods of time necessary, to achieve the desired therapeutic or prophylactic result. However, “effective amount” of a substance/molecule of the present disclosure may vary according to factors such as the disease state, age, sex, and weight of the individual, and the ability of the substance/molecule, to elicit a desired response in the individual. An effective amount is also one in which any toxic or detrimental effects of the substance/molecule are outweighed by the therapeutically beneficial effects. In one embodiment the effective amount of the anti-ENO-1 antibody ranges from 1-1000 mg/kg, preferably 5-100 mg/kg, more preferably 10-50 mg/kg, e.g. 5, 10, 15, 20, 30, 40, 50, 60, 70, 80, 90 or 100 mg/kg.

The term “therapeutic agent” as used herein refers to a substance that inhibits or prevents the function of cells and/or causes destruction of cells. The term is intended to include an anti-fibrotic agent, radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, ⁶⁰C, and radioactive isotopes of lutetium-177, strontium-89 and samarium (¹⁵³Sm)), immunomodulator, and toxins such as small molecule toxins or enzymatically active toxins of bacterial, fungal, plant or animal origin, including synthetic analogs and derivatives thereof.

A “fibrotic condition,” “fibroproliferative condition,” “fibrotic disease,” “fibroproliferative disease,” “fibrotic disorder,” and “fibroproliferative disorder” are used interchangeably to refer to a condition, disease or disorder that is characterized by dysregulated proliferation or activity of fibroblasts and/or pathologic or excessive accumulation of collagenous tissue. Typically, any such disease, disorder or condition is amenable to treatment by administration of a compound having anti-fibrotic effects. Fibrotic disease include, but are not limited to, idiopathic pulmonary fibrosis (IPF), pulmonary hypertension, pulmonary fibrosis, emphysema, nonalcoholic steatohepatitis, pancreatic fibrosis, intestinal fibrosis, cardiac fibrosis, myelofibrosis, arthrofibrosis, interstitial lung diseases, non-specific interstitial pneumonia (NSIP), usual interstitial pneumonia (UIP), endomyocardial fibrosis, mediastinal fibrosis, retroperitoneal fibrosis, progressive massive fibrosis (a complication of coal workers' pneumoconiosis), nephrogenic systemic fibrosis, Crohn's disease, old myocardial infarction, scleroderma/systemic sclerosis, neurofibromatosis, Hermansky-Pudlak syndrome, diabetic nephropathy, renal fibrosis, hypertrophic cardiomyopathy (HCM), hypertension-related nephropathy, focal segmental glomerulosclerosis (FSGS), radiation-induced fibrosis, uterine leiomyomas (fibroids), alcoholic liver disease, hepatic steatosis, hepatic fibrosis, hepatic cirrhosis, hepatitis C virus (HCV) infection, chronic organ transplant rejection, fibrotic conditions of the skin, keloid scarring, Dupuytren contracture, Ehlers-Danlos syndrome, epidermolysis bullosa dystrophica, oral submucous fibrosis, or fibro-proliferative disorders.

The term “idiopathic pulmonary fibrosis” as used herein refers to a chronic, progressive, and usually lethal lung disorder, thought to be a consequence of a chronic inflammatory process.

The term “anti-fibrotic agent” as used herein refers to a substance that is known to have anti-fibrotic effects. The term is intended to include pirfenidone or receptor tyrosine kinase inhibitors (RTKIs) such as Nintedanib, Sorafenib and other RTKIs, or angiotensin II (AT1) receptor blockers, or CTGF inhibitor, or any antifibrotic compound susceptible to interfere with the TGF-β and BMP-activated pathways including activators of the latent TGF-β complex such as MMP2, MMP9, THBS1 or cell-surface integrins, TGF3 receptors type I (TGFBRI) or type II (TGFBRII) and their ligands such as TGF3, Activin, inhibin, Nodal, anti-Mijllerian hormone, GDFs or BMPs, auxiliary co-receptors (also known as type III receptors), or components of the SMAD-dependent canonical pathway including regulatory or inhibitory SMAD proteins, or members of the SMAD-independent or non-canonical pathways including various branches of MAPK signaling, TAK1, Rho-like GTPase signaling pathways, phosphatidylinositol-3 kinase/AKT pathways, TGF-β-induced EMT process, or canonical and non-canonical Hedgehog signaling pathways including Hh ligands or target genes, or any members of the WNT, or Notch pathways which are susceptible to influence TGF-β signaling.

The medicaments of the present disclosure may be applied locally or systemically. The medicaments of the present disclosure may also be supplied in combinations or with cofactors. Medicaments of the present disclosure may be administered in an amount sufficient to restore normal levels, if the medicament of the present disclosure is normally present in the target location, or they may be administered in an amount to raise levels above normal levels in the target location.

The medicaments of the present disclosure may be supplied to a target location from an exogenous source, or they may be made in vivo by cells in the target location or cells in the same organism as the target location.

Medicaments of the present disclosure may be in any physiologically appropriate formulation. They may be administered to an organism by injection, topically, by inhalation, orally or by any other effective means.

The same medicaments and methodologies described above to suppress or inhibit excessive fibrosis formation and maintenance may also be used to suppress or inhibit inappropriate fibrosis formation. For example, they may treat or prevent a condition occurring in the liver, kidney, lung, heart and pericardium, eye, skin, mouth, pancreas, gastrointestinal tract, brain, breast, bone marrow, bone, genitourinary, a tumor, or a wound.

Generally, they may treat or prevent fibrosis resulting from conditions including but not limited to rheumatoid arthritis, lupus, pathogenic fibrosis, fibrosing disease, fibrotic lesions such as those formed after Schistosoma japonicum infection, radiation damage, autoimmune diseases, lyme disease, chemotherapy induced fibrosis, HIV or infection-induced focal sclerosis, failed back syndrome due to spinal surgery scarring, abdominal adhesion post-surgery scarring, and fibrocystic formations.

Embodiments of the present disclosure relate to antibody-drug conjugates containing ENO-1 antibodies and their uses in in treating a fibrotic disease. ENO-1 is a multiple functional protein, which is found to express on the cell surfaces of many cancer cells as a plasminogen receptor and on activated hematopoietic cells, such as neutrophils, lymphocytes, and monocytes. Therefore, ADCs based on antibodies against ENO-1 can be useful diagnostic and/or treatment agents.

However, the fast internalization or lacking ADCC activity of therapeutic antibody might result in antibody ineffective as well as resistance. Therefore, there is a need to enhance the therapeutic efficacy of anti-ENO-1 based therapeutics. One approach is to conjugate a payload with an anti-ENO-1 antibody (i.e., an antibody-drug conjugate).

In accordance with embodiments of the present disclosure, anti-ENO-1 antibodies, or a binding fragment thereof, may be coupled to a drug, diagnostic agent, or a therapeutic agent. Thus, the term “antibody-drug conjugate” (ADC) as used herein may refer to an antibody portion (which can be a whole antibody or a binding fragment thereof) coupled to a payload (which can be a drug, a diagnostic agent or a therapeutic agent). Exemplary ADC is as described in WO 2021/228044 A1, the contents of which are incorporated by reference in its entirety.

### Methods of Treating a Fibrotic Disease

The present disclosure provides methods of treating a fibrotic disease, e.g. idiopathic pulmonary fibrosis to (IPF). The methods generally involve administering to a subject in need thereof an effective amount of alpha-enolase (enolase-1, ENO-1) antagonist.

In some embodiments, the ENO-1 antagonist used in the method may include, but not limited to:
(1) an anti-ENO-1 antibody or the binding fragment thereof;
(2) an anti-ENO-1 chimeric antigen receptor (CAR), comprising an antigen binding domain, a hinge region, a transmembrane domain, and a signaling domain; wherein the antigen binding domain is at least a portion of anti-ENO-1 antibody;
(3) an immunoconjugate that binds specifically to ENO-1, comprising: the general formula of Ab-(L-D)m (I), wherein Ab is an anti-ENO-1 antibody or the binding fragment thereof, L is a linker or a direct bond, D is a therapeutic agent or a label, and m is an integer from 1 to 12; or
(4) nucleic acid of anti-ENO-1 antibody or the binding fragment thereof or anti-ENO-1 CAR, which is delivered into cells and expressed as intracellular anti-ENO-1 antibody or the binding fragment thereof or anti-ENO-1 CAR.

Embodiments of the present disclosure will be illustrated with the following specific examples. One skilled in the art would appreciate that these examples are for illustration only and that other modifications and variations are possible without departing from the scope of the present disclosure.

### EXAMPLES

### Example 1. Preparation of Anti-ENO-1 Antibody

In accordance with embodiments of the present disclosure, a general method for the generation of anti-ENO-1 antibodies includes obtaining a hybridoma producing a monoclonal antibody against ENO-1. Methods to produce monoclonal antibodies are known in the art and will not be elaborated here. Briefly, mice are challenged with antigen (ENO-1) with an appropriate adjuvant. Then, the spleen cells of the immunized mice were harvested and fused with hybridoma. Positive clones may be identified for their abilities to bind ENO-1 antigen, using any known methods, such as ELISA. In an embodiment, the anti-ENO-1 antibody is HuL217.

Antibody-drug conjugates (ADCs) can specifically target ENO-1. These ADCs can use any antibody that binds specifically to ENO-1. For example, ADCs of the claimed invention may use a mouse or humanized anti-ENO-1 antibody, or a scFv or Fab fragment thereof. An exemplary anti-ENO-1 antibody, e.g. HuL217, may comprise a heavy-chain variable domain having three complementary regions including HCDR1 (GYTFTSCVMN; SEQ ID NO: 1), HCDR2 (YINPYNDGTKYNEKFKG; SEQ ID NO: 2) and HCDR3 (EGFYYGNFDN; SEQ ID NO: 3), and a light-chain variable domain having three complementary regions including LCDR1 (RASENIYSYLT; SEQ ID NO: 4), LCDR2 (NAKTLPE; SEQ ID NO: 5) and LCDR3 (QHHYGTPYT; SEQ ID NO: 6). An another exemplary anti-ENO-1 antibody may comprise a heavy-chain variable domain having three complementary regions including HCDR1 (GYTFTSXVMN, wherein X is any amino acid but cysteine; SEQ ID NO: 7), HCDR2 (YINPYNDGTKYNEKFKG; SEQ ID NO: 2) and HCDR3 (EGFYYGNFDN; SEQ ID NO: 3), and a light-chain variable domain having three complementary regions including LCDR1 (RASENIYSYLT; SEQ ID NO: 4), LCDR2 (NAKTLPE; SEQ ID NO: 5) and LCDR3 (QHHYGTPYT; SEQ ID NO: 6).

In accordance with embodiments of the present disclosure, the antibodies may be mouse antibodies. Alternatively, the antibodies may be chimeric antibodies (e.g., human constant regions coupled to the mouse variable regions) or humanized antibodies (e.g., mouse CDRs grafted on the framework regions of human immunoglobulins) or completely human antibodies.

The monoclonal antibody may be humanized by obtaining the CDR sequences from the hybridoma and cloning the CDR sequences into human framework sequences to produce humanized antibodies. Any common methods known in the art for identifying CDR sequences may be used. The CDR regions in this invention are identified with the Kabat number scheme. First, a hybridoma of anti-ENO-1 (e.g., mouse hybridoma) was generated. Such a hybridoma may be generated with standard protocols to produce monoclonal antibodies. The total RNA of the hybridoma was then isolated, for example using the TRIzol® reagent. Then, cDNA was synthesized from the total RNA, for example using a first strand cDNA synthesis kit (Superscript III) and an oligo(dT₂₀) primer or an Ig-3′ constant region primer.

Heavy and light chain variable regions of the immunoglobulin genes were then cloned from the cDNA. For example, the VH and VL variable regions of the anti-ENO-1 mAb were amplified from mouse hybridoma cDNAs by PCR, using a mouse Ig-5′ primer set (Novagen). The PCR products may be cloned directly into a suitable vector (e.g., a pJET1.2 vector) using CloneJet™ PCR Cloning Kit (Ferments). The pJET1.2 vector contains lethal insertions and will survive the selection conditions only when the desired gene is cloned into this lethal region. This facilitates the selection of recombinant colonies. Finally, the recombinant colonies were screened for the desired clones, the DNAs of those clones were isolated and sequenced. The immunoglobulin (IG) nucleotide sequences may be analyzed at the international ImMunoGeneTics information system (IGMT) website.

### Antibody Expression and Purification

For antibody production, the isolated clones may be expressed in any suitable cells. As an example, F293 cells (Life technologies) were transfected with the anti-ENO-1 mAb expressing plasmid and cultured for 7 days. The anti-ENO-1 antibody was purified from the culture medium using a protein A affinity column (GE). Protein concentrations may be determined with a Bio-Rad protein assay kit and analyzed with 12% SDS-PAGE, using procedures known in the art or according to the manufacturer's instructions.

### Example 2. Overexpression of ENO-1 in Human and Mouse Fibrotic Lungs

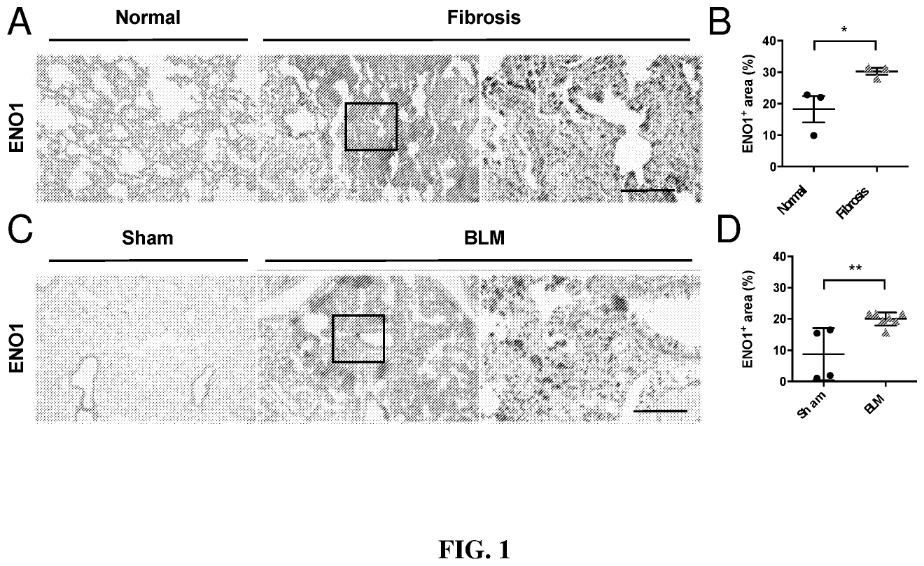

In this example, ENO-1 immunohistochemistry (IHC) staining was used to determine whether ENO-1 is abnormally expressed in fibrotic lungs. In FIG. 1A, formalin-fixed, paraffin-embedded (FFPE) lung tissue samples were purchased from commercial sources. Three normal human lung FFPE tissue sections were obtained from BioChain Institute, Inc. (Newark, CA, USA) and US Biomax (Derwood, MD, USA). Three human fibrotic lung FFPE slides were obtained from OriGene Technologies (Rockville, MD, USA). ENO-1 expression was found to be elevated in human fibrotic lungs but not in normal lungs. Quantitative results were shown in FIG. 1B.

Bleomycin-induced pulmonary fibrosis in C57BL/6 mice which is the most commonly used experimental model for the human fibrotic disease, idiopathic pulmonary fibrosis. 7 to 9-week-old male C57BL/6 mice were intra-tracheally given with single dosing of bleomycin (3 mg/kg). After 21 days, lungs were harvested for ENO-1 IHC staining. FIG. 1C illustrates ENO-1 is overexpressed in the bleomycin group (BLM) compared to sham group. Quantitative results were shown in FIG. 1D

### Example 3. IPF Disease Model of ENO-1 Antibody

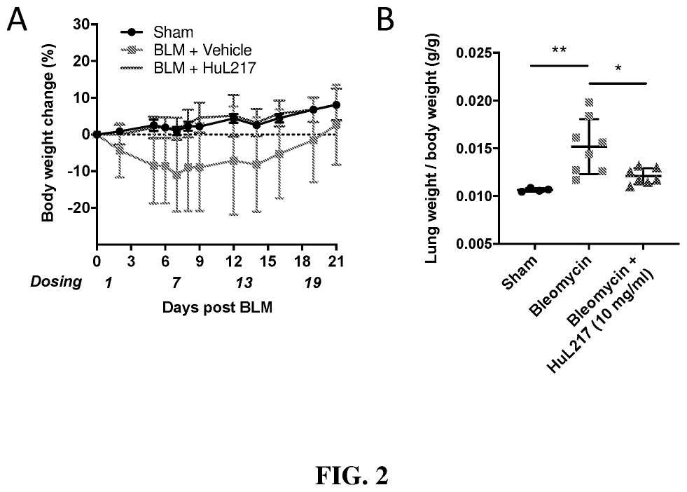

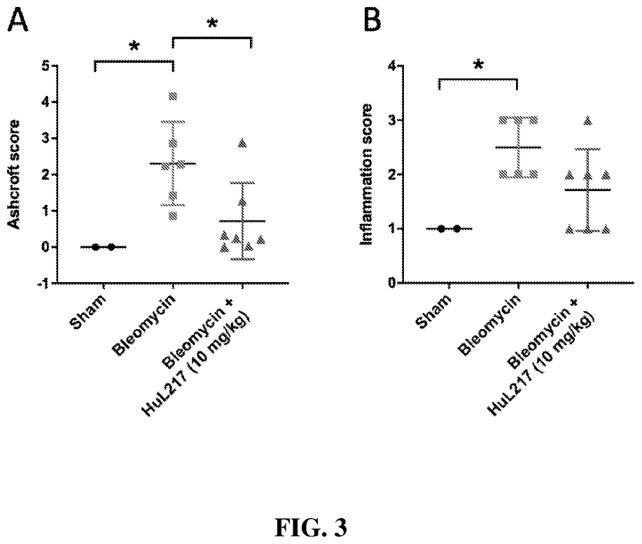

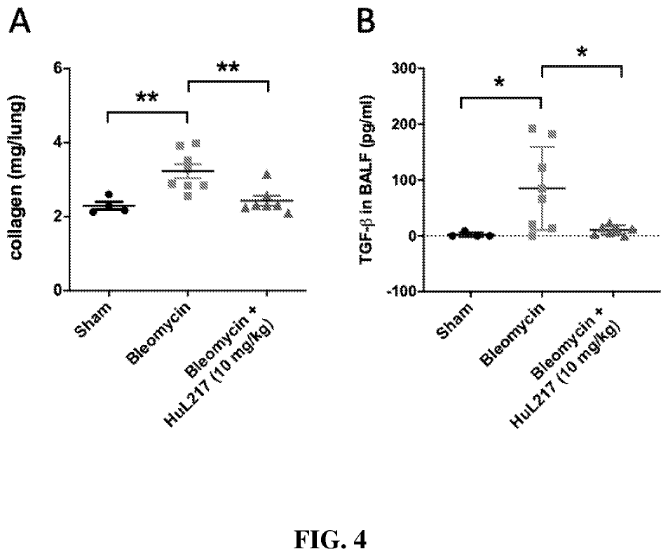

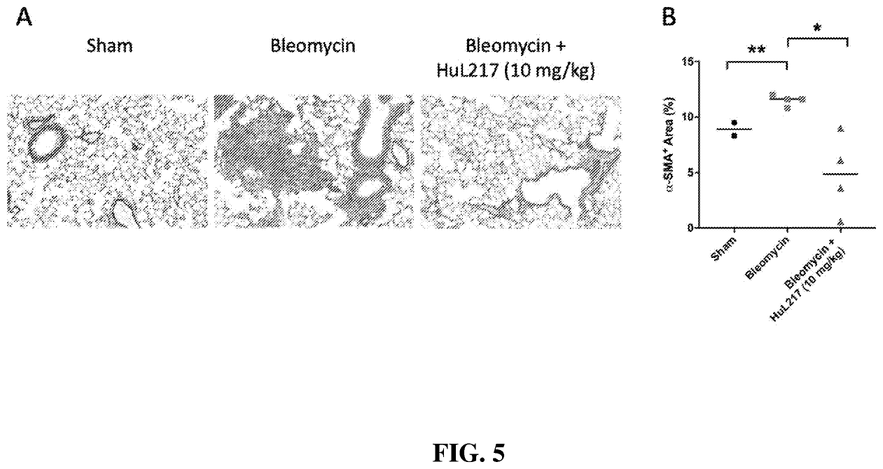

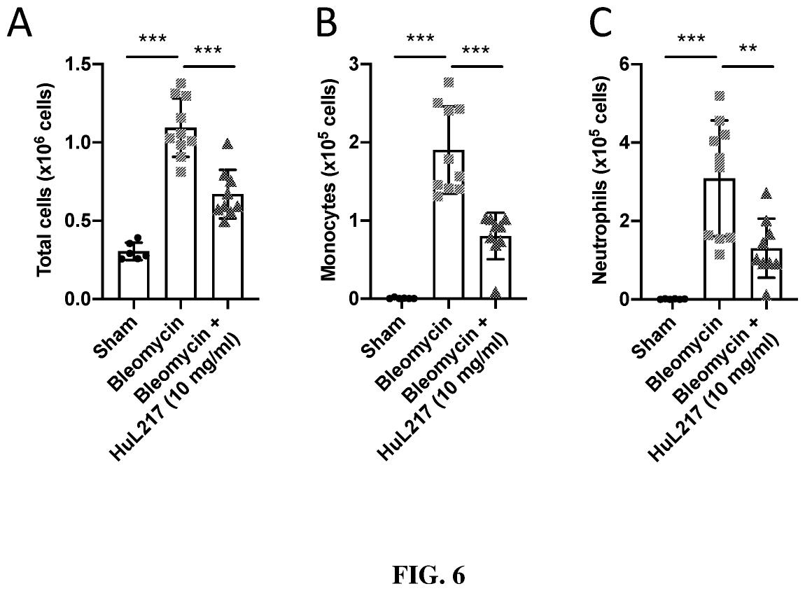

HuL217 was evaluated in bleomycin-induced pulmonary fibrosis in C57BL/6 mice. 7 to 9-week-old male C57BL/6 mice were intra-tracheally given with single dosing of bleomycin (3 mg/kg). Mice were randomly divided into 3 groups with 4 mice in sham group, 7 mice in Bleomycin+Vehicle or Bleomycin+HuL217 groups, respectively. The day of bleomycin challenge was set as day 0. Mice of HuL217 group were injected intravenously on day 1, 7, 13, and 19. Results illustrates treating with HuL217 was able to attenuate body weight loss and lung weight gain (FIG. 2), Ashcroft and inflammation scores (FIG. 3), and lung collagen content and TGF-β levels in bronchial alveolar lavage fluid (BALF) (FIG. 4), as compared with the bleomycin group. Furthermore, accumulation of myofibroblasts in the lungs of bleomycin-induced mice was reduced by HuL217 treatment (FIG. 5). To investigate the anti-inflammatory effect of HuL217, recruitment of monocytes and neutrophils into the lungs was analyzed in BALF by using flow cytometry. FIG. 6 demonstrated HuL217 (intravenously injected 2 hours prior to bleomycin injection) reduced the recruitment of monocytes and neutrophils into the lungs of bleomycin-induced mice on day 4.

### Example 4. In Vitro Effects of ENO-1 Antibody on Lung Fibroblasts

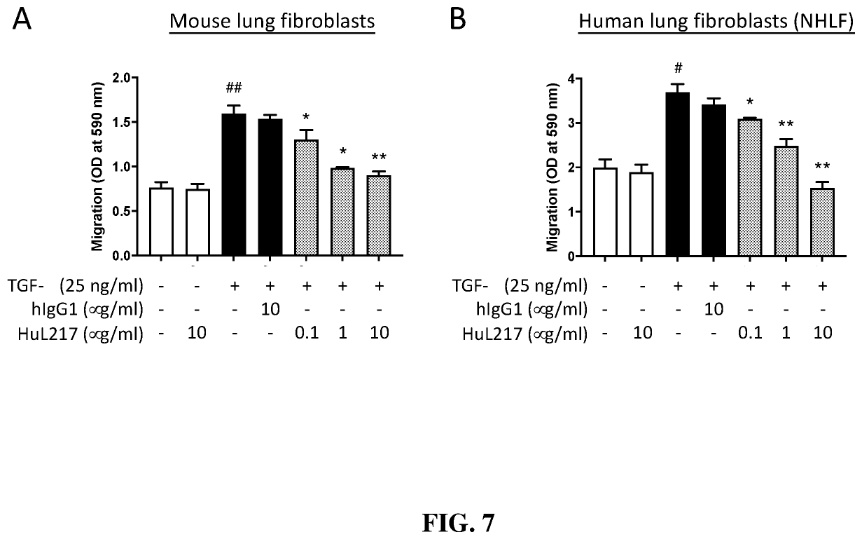

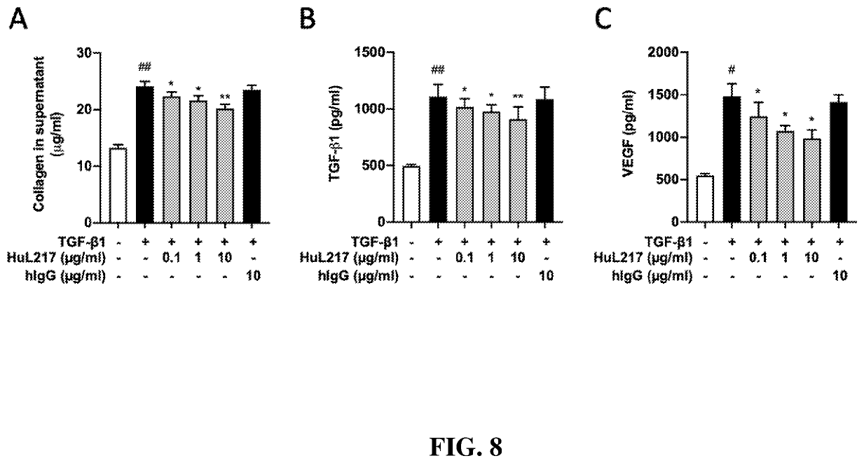

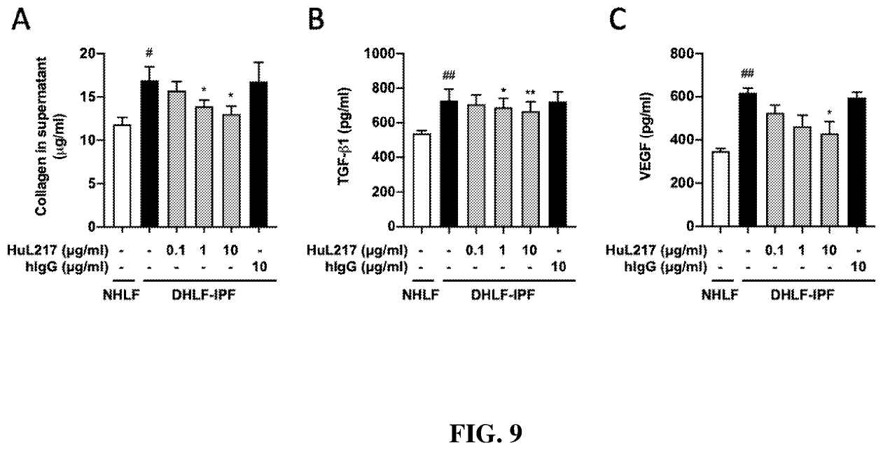

Primary mouse lung fibroblasts were isolated from male C57BL/6 mice and primary human lung fibroblasts were purchased from Lonza (London, UK). Both cells were treated with TGF-β to induce migration. FIG. 7 demonstrated HuL217 dose-dependently reduced the migration ability of TGF-β-treated primary lung fibroblasts. In vitro anti-fibrotic effects of HuL217 were further demonstrated by using primary human lung fibroblasts isolated from normal (NHLF) or IPF patients (DHLF-IPF). HuL217 could dose-dependently reduced secretion of collagen, TGF-01, and VEGF in either TGF-β-simulated NHLF (FIG. 8) or DHLF-IPF (FIG. 9).

In summary, in bleomycin mice, HuL217 significantly attenuated body weight loss and lung weight gain as well as the fibrosis lesion and collagen deposition in lungs. The elevated amount of TGF-β and monocytes were also reduced in BALF. HuL217 could significantly reduce cell migration and secretion of collagen and TGF-β in primary mouse lung myofibroblasts.

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

All references cited herein are incorporated herein by reference to the full extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

What is claimed is:

1. Use of an effective amount of alpha-enolase (enolase-1, ENO-1) antagonist in manufacturing a medicament for treating a fibrotic disease.

2. The use of claim 1, wherein the ENO-1 antagonist is an anti-ENO-1 antibody or the binding fragment thereof.

3. The use of claim 1, wherein the ENO-1 antagonist is an anti-ENO-1 chimeric antigen receptor (CAR), comprising an antigen binding domain, a hinge region, a transmembrane domain, and a signaling domain; wherein the antigen binding domain is at least a portion of anti-ENO-1 antibody.

4. The use of claim 3, wherein the antigen binding domain comprises the amino acid sequences shown in SEQ ID NO: 1 (GYTFTSCVMN), SEQ ID NO: 2 (YINPYNDGTKYNEKFKG), SEQ ID NO: 3 (EGFYYGNFDN), SEQ ID NO: 4 (RASENIYSYLT), SEQ ID NO: 5 (NAKTLPE) and SEQ ID NO: 6 (QHHYGTPYT).

5. The use of claim 3, the antigen binding domain comprises the amino acid sequences shown in SEQ ID NO: 7 (GYTFTSXVMN, wherein X is any amino acid but cysteine), SEQ ID NO: 2 (YINPYNDGTKYNEKFKG), SEQ ID NO: 3 (EGFYYGNFDN), SEQ ID NO: 4 (RASENIYSYLT), SEQ ID NO: 5 (NAKTLPE) and SEQ ID NO: 6 (QHHYGTPYT).

6. The use of any one of claims 3-5, wherein the hinge region comprises a CD8 alpha hinge region (TTTPAPRPPTPAPTIASQPLSLRPEACRPAAGGAVHT RGLDFACD; SEQ ID NO: 8);
optionally, the transmembrane domain comprises a CD8 alpha transmembrane region (IYIWAPLAGTCGVLLLSLVIT; SEQ ID NO: 9) and/or a CD28 transmembrane region (FWVLVVVGGVLACYSLLVTVAFIIFWV; SEQ ID NO: 10);
optionally, the signaling domain comprises CD3 zeta
SEQ ID NO: 11
(RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR;);
optionally, the signaling domain further comprises 4-1BB, the intracellular region of CD28, DAP10, OX 40 or a combination thereof;
optionally, the anti-ENO-1 CAR further comprises a signal peptide;
optionally, the signal peptide comprises an IgG kappa light chain signal peptide, a CD8 alpha signal peptide, a GM-CSF signal peptide, an HSA signal peptide, an IgG heavy chain signal peptide, an IgG light chain signal peptide, a CD33 signal peptide, an IL-2 signal peptide, or an insulin signal peptide.

7. The use of any one of claims 3-5, wherein the anti-ENO-1 CAR comprises a signal peptide, an anti-ENO-1 antibody, a CD8 alpha hinge region, a CD8 alpha transmembrane region, 4-1BB, and CD3 zeta.

8. The use of claim 1, wherein the ENO-1 antagonist is an immunoconjugate that binds specifically to ENO-1, comprising:
the general formula of Ab-(L-D)m (I),
wherein Ab is an anti-ENO-1 antibody or the binding fragment thereof, L is a linker or a direct bond, D is a therapeutic agent or a label, and m is an integer from 1 to 12.

9. The use of claim 8, wherein the antibody is a monoclonal antibody.

10. The use of claim 8, wherein the antibody is a mouse antibody, a human antibody, a chimeric antibody, a humanized antibody, or an antibody fragment thereof.

11. The use of claim 8, wherein the therapeutic agent comprises an anti-fibrotic agent, immunomodulator, radioactive isotopes, and toxins.

12. The use of claim 8, wherein the anti-fibrotic agent comprises pirfenidone or receptor tyrosine kinase inhibitors (RTKIs) such as Nintedanib, Sorafenib and other RTKIs, or angiotensin II (AT1) receptor blockers, or CTGF inhibitor, or any antifibrotic compound susceptible to interfere with the TGF-β and BMP-activated pathways including activators of the latent TGF-β complex such as MMP2, MMP9, THBS1 or cell-surface integrins, TGF3 receptors type I (TGFBRI) or type II (TGFBRII) and their ligands such as TGF3, Activin, inhibin, Nodal, anti-Mijllerian hormone, GDFs or BMPs, auxiliary co-receptors (also known as type III receptors), or components of the SMAD-dependent canonical pathway including regulatory or inhibitory SMAD proteins, or members of the SMAD-independent or non-canonical pathways including various branches of MAPK signaling, TAK1, Rho-like GTPase signaling pathways, phosphatidylinositol-3 kinase/AKT pathways, TGF-β-induced EMT process, or canonical and non-canonical Hedgehog signaling pathways including Hh ligands or target genes, or any members of the WNT, or Notch pathways which are susceptible to influence TGF-β signaling.

13. The use of claim 8, wherein the label comprises a diagnostic or imaging reagent.

14. The use of claim 8, wherein the antibody comprises
a heavy-chain variable domain having three complementary regions including
HCDR1 (GYTFTSCVMN; SEQ ID NO: 1),
HCDR2 (YINPYNDGTKYNEKFKG; SEQ ID NO: 2), and
HCDR3 (EGFYYGNFDN; SEQ ID NO: 3); and
a light-chain variable domain having three complementary regions including
LCDR1 (RASENIYSYLT; SEQ ID NO: 4),
LCDR2 (NAKTLPE; SEQ ID NO: 5), and
LCDR3 (QHHYGTPYT; SEQ ID NO: 6).

15. The use of claim 8, wherein the antibody comprises
a heavy-chain variable domain having three complementary regions including
HCDR1 (GYTFTSXVMN, wherein X is any amino acid but cysteine; SEQ ID NO: 7),
HCDR2 (YINPYNDGTKYNEKFKG; SEQ ID NO: 2), and
HCDR3 (EGFYYGNFDN; SEQ ID NO: 3); and
a light-chain variable domain having three complementary regions including
LCDR1 (RASENIYSYLT; SEQ ID NO: 4),
LCDR2 (NAKTLPE; SEQ ID NO: 5), and
LCDR3 (QHHYGTPYT; SEQ ID NO: 6).

16. The use of claim 1, wherein the ENO-1 antagonist is nucleic acid to be delivered into cells and expressed as intracellular protein or peptide.

17. The use of claim 16, wherein the ENO1 antagonist is secreted, non-secreted, or a combination thereof.

18. The use of claim 16, wherein the ENO-1 antagonist is delivered via a viral vector, a polymer, and/or liposome.

19. The use of claim 1, wherein the fibrotic diseases comprises idiopathic pulmonary fibrosis (IPF), pulmonary hypertension, pulmonary fibrosis, emphysema, nonalcoholic steatohepatitis, pancreatic fibrosis, intestinal fibrosis, cardiac fibrosis, myelofibrosis, arthrofibrosis, interstitial lung diseases, non-specific interstitial pneumonia (NSIP), usual interstitial pneumonia (UIP), endomyocardial fibrosis, mediastinal fibrosis, retroperitoneal fibrosis, progressive massive fibrosis (a complication of coal workers' pneumoconiosis), nephrogenic systemic fibrosis, Crohn's disease, old myocardial infarction, scleroderma/systemic sclerosis, neurofibromatosis, Hermansky-Pudlak syndrome, diabetic nephropathy, renal fibrosis, hypertrophic cardiomyopathy (HCM), hypertension-related nephropathy, focal segmental glomerulosclerosis (FSGS), radiation-induced fibrosis, uterine leiomyomas (fibroids), alcoholic liver disease, hepatic steatosis, hepatic fibrosis, hepatic cirrhosis, hepatitis C virus (HCV) infection, chronic organ transplant rejection, fibrotic conditions of the skin, keloid scarring, Dupuytren contracture, Ehlers-Danlos syndrome, epidermolysis bullosa dystrophica, oral submucous fibrosis, or fibro-proliferative disorders.

20. A method for treating a fibrotic disease, comprising:
administering to a subject in need thereof an effective amount of alpha-enolase (enolase-1, ENO-1) antagonist.

21. The method of claim 20, wherein the fibrotic diseases comprises idiopathic pulmonary fibrosis (IPF), pulmonary hypertension, pulmonary fibrosis, emphysema, nonalcoholic steatohepatitis, pancreatic fibrosis, intestinal fibrosis, cardiac fibrosis, myelofibrosis, arthrofibrosis, interstitial lung diseases, non-specific interstitial pneumonia (NSIP), usual interstitial pneumonia (UIP), endomyocardial fibrosis, mediastinal fibrosis, retroperitoneal fibrosis, progressive massive fibrosis (a complication of coal workers' pneumoconiosis), nephrogenic systemic fibrosis, Crohn's disease, old myocardial infarction, scleroderma/systemic sclerosis, neurofibromatosis, Hermansky-Pudlak syndrome, diabetic nephropathy, renal fibrosis, hypertrophic cardiomyopathy (HCM), hypertension-related nephropathy, focal segmental glomerulosclerosis (FSGS), radiation-induced fibrosis, uterine leiomyomas (fibroids), alcoholic liver disease, hepatic steatosis, hepatic fibrosis, hepatic cirrhosis, hepatitis C virus (HCV) infection, chronic organ transplant rejection, fibrotic conditions of the skin, keloid scarring, Dupuytren contracture, Ehlers-Danlos syndrome, epidermolysis bullosa dystrophica, oral submucous fibrosis, or fibro-proliferative disorders.

22. ENO-1 antagonist of any one of claims 1-18 for use in treating a fibrotic disease.

23. The ENO-1 antagonist for use of claim 22, wherein the fibrotic diseases comprises idiopathic pulmonary fibrosis (IPF), pulmonary hypertension, pulmonary fibrosis, emphysema, nonalcoholic steatohepatitis, pancreatic fibrosis, intestinal fibrosis, cardiac fibrosis, myelofibrosis, arthrofibrosis, interstitial lung diseases, non-specific interstitial pneumonia (NSIP), usual interstitial pneumonia (UIP), endomyocardial fibrosis, mediastinal fibrosis, retroperitoneal fibrosis, progressive massive fibrosis (a complication of coal workers' pneumoconiosis), nephrogenic systemic fibrosis, Crohn's disease, old myocardial infarction, scleroderma/systemic sclerosis, neurofibromatosis, Hermansky-Pudlak syndrome, diabetic nephropathy, renal fibrosis, hypertrophic cardiomyopathy (HCM), hypertension-related nephropathy, focal segmental glomerulosclerosis (FSGS), radiation-induced fibrosis, uterine leiomyomas (fibroids), alcoholic liver disease, hepatic steatosis, hepatic fibrosis, hepatic cirrhosis, hepatitis C virus (HCV) infection, chronic organ transplant rejection, fibrotic conditions of the skin, keloid scarring, Dupuytren contracture, Ehlers-Danlos syndrome, epidermolysis bullosa dystrophica, oral submucous fibrosis, or fibro-proliferative disorders.


SEQUENCE LISTING (11 sequences)

SEQ ID NO: 1 - protein, 10 aa, synthetic construct
  REGION 1..10: note = HCDR1
GYTFTSCVMN

SEQ ID NO: 2 - protein, 17 aa, synthetic construct
  REGION 1..17: note = HCDR2
YINPYNDGTKYNEKFKG

SEQ ID NO: 3 - protein, 10 aa, synthetic construct
  REGION 1..10: note = HCDR3
EGFYYGNFDN

SEQ ID NO: 4 - protein, 11 aa, synthetic construct
  REGION 1..11: note = LCDR1
RASENIYSYLT

SEQ ID NO: 5 - protein, 7 aa, synthetic construct
  REGION 1..7: note = LCDR2
NAKTLPE

SEQ ID NO: 6 - protein, 9 aa, synthetic construct
  REGION 1..9: note = LCDR3
QHHYGTPYT

SEQ ID NO: 7 - protein, 10 aa, synthetic construct
  REGION 1..10: note = HCDR1
  MUTAGEN 7: note = Xaa is any amino acid but cysteine
GYTFTSXVMN

SEQ ID NO: 8 - protein, 45 aa, synthetic construct
TTTPAPRPPTPAPTIASQPLSLRPEACRPAAGGAVHTRGLDFACD

SEQ ID NO: 9 - protein, 21 aa, synthetic construct
IYIWAPLAGTCGVLLLSLVIT

SEQ ID NO: 10 - protein, 27 aa, synthetic construct
FWVLVVVGGVLACYSLLVTVAFIIFWV

SEQ ID NO: 11 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR